(19) United States
(12) Patent Application Publication
(10) Pub. No.: US 2022/0361845 A1
(43) Pub. Date: Nov. 17, 2022

(54) ULTRASOUND ENDOSCOPE

(71) Applicant: FUJIFILM Corporation, Tokyo (JP)

(72) Inventors: Noboru KINOMOTO, Kanagawa (JP); Yasuhiko MORIMOTO, Kanagawa (JP)

(73) Assignee: FUJIFILM Corporation, Tokyo (JP)

(21) Appl. No.: 17/735,103

(22) Filed: May 2, 2022

(30) Foreign Application Priority Data
May 13, 2021 (JP) 2021-081487

(51) Int. Cl. A61B 8/00; A61B 8/12

(52) U.S. Cl. CPC A61B 8/445; A61B 8/12

(57) ABSTRACT

Provided is an ultrasound endoscope capable of achieving both improvement of the kink resistance of a forceps tube in a bending part and improvement of electromagnetic wave shieldability in a distal end hard part.

A forceps tube is configured such that a metal element wire is wound around a tube bending part that is disposed inside a bending part, and a tube distal end part including an opening portion facing region facing an opening portion of a shield ring is formed of a material that is hardly influenced by electromagnetic waves. The tube distal end part is configured of only the forceps tube. That is, the metal element wire that is influenced by electromagnetic waves is not wound around the tube distal end part, and resin, such as fluororubber or silicon rubber, to be hardly influenced by electromagnetic waves is exposed from the opening portion facing region.

### CROSS-REFERENCE TO RELATED APPLICATION

The present application claims priority under 35 U.S.C § 119(a) to Japanese Patent Application No. 2021-081487 filed on May 13, 2021, which is hereby expressly incorporated by reference, in its entirety, into the present application.

### BACKGROUND OF THE INVENTION

### 1. Field of the Invention

The present invention relates to an ultrasound endoscope, and in particular, an ultrasound endoscope having an ultrasound oscillator and a treatment tool outlet port in a distal end part of an insertion part.

### 2. Description of the Related Art

In recent years, an ultrasound endoscope is used in a medical field. The ultrasound endoscope comprises an observation system that captures an image inside a body of a subject, and an ultrasound probe that irradiates the inside of the body of the subject with ultrasonic waves and receives reflected waves to capture video. In such an ultrasound endoscope, for example, as disclosed in JP2008-237842A, a plurality of ultrasound oscillators of the ultrasound probe are electrically connected to a plurality of signal wires for ultrasonic waves, respectively.

In the ultrasound endoscope of JP2008-237842A, a forceps pipe is disposed inside a distal end hard part where the ultrasound probe is provided. A distal end side of the forceps pipe is connected to a treatment tool leading-out opening portion formed in a distal end surface of the distal end hard part, and a proximal end side of the forceps pipe is connected to a forceps tube.

### SUMMARY OF THE INVENTION

Incidentally, a bending part of the endoscope is operated in an up-down direction and a right-left direction, and the operation is repeated. Accordingly, a load is repeatedly applied to the forceps tube and damage (kink) may occur. As a countermeasure, for example, it is considered that a metallic member is wound around an outer peripheral surface of the forceps tube, thereby improving the kink resistance of the forceps tube in the bending part of the endoscope.

Note that, in the ultrasound endoscope, since the metallic member disposed near an ultrasound transducer may become an electromagnetic wave transmission source or an electromagnetic wave reception source due to an influence of electromagnetic waves emitted from the ultrasound transducer, in this case, there is a problem that electromagnetic compatibility (EMC) performance is degraded. For this reason, it is not possible to dispose the forceps tube with the metallic member wound therearound near the ultrasound transducer, and it is difficult to improve the kink resistance of the forceps tube while avoiding the influence of the electromagnetic waves. While it is also considered that a metallic member is incorporated inside an outer shell of the forceps tube, there is a limit in suppressing the influence of the electromagnetic waves.

In this way, in the ultrasound endoscope, it is difficult to solve both problems of improvement of the kink resistance of the forceps tube in the bending part and improvement of electromagnetic wave shieldability in the distal end hard part.

The present invention has been accomplished in view of such a situation, and an object of the present invention is to provide an ultrasound endoscope capable of achieving both improvement of the kink resistance of a forceps tube in a bending part and improvement electromagnetic wave shieldability in a distal end hard part.

To attain the above-described object, the present invention provides an ultrasound endoscope comprising an insertion part in which a distal end hard part, a bending part connected to a proximal end side of the distal end hard part, and a soft part connected to a proximal end side of the bending part are provided along a longitudinal axis direction, an ultrasound transducer in which a plurality of ultrasound oscillators configured to transmit and receive ultrasonic waves are arranged along a peripheral direction of the distal end hard part, a forceps channel that is inserted into the insertion part and has a distal end side opened on a distal end surface of the distal end hard part, a shield member that is disposed between the ultrasound transducer and the forceps channel, and suppresses electromagnetic waves emitted from the ultrasound transducer, an ultrasonic wave shielded cable that passes through the bending part from the soft part and has a distal end part disposed on one side to be a side of the shield member on which the forceps channel is disposed, and a plurality of signal wires that are accommodated in the ultrasonic wave shielded cable, extend from the distal end part of the ultrasonic wave shielded cable, and are connected to the plurality of ultrasound oscillators, respectively. The shield member has an opening portion for wiring the plurality of signal wires from the one side of the shield member to the other side to be a side on which the ultrasound transducer is disposed, the forceps channel has a metallic forceps pipe disposed on the one side of the shield member, and a forceps tube that is connected to a proximal end side of the forceps pipe on the one side of the shield member, and the forceps tube is configured such that a metal element wire is wound around at least a part inside the bending part, and a tube distal end part including a region facing the opening portion of the shield member is formed of a material that is hardly influenced by the electromagnetic waves.

According to a form of the present invention, it is preferable that the metal element wire is provided in only other parts in the forceps tube except for the tube distal end part.

According to a form of the present invention, it is preferable that bending rigidity of the tube distal end part is higher than bending rigidity of the other parts.

According to a form of the present invention, it is preferable that the forceps tube is provided with grooves for winding the metal element wire around the tube distal end part and the other parts, and the groove provided in the tube distal end part is shallower than a depth of the groove provided in the other parts.

According to a form of the present invention, it is preferable that, in the forceps tube, a groove for winding the metal element wire is provided in only the other parts.

According to a form of the present invention, it is preferable that a thickness of the tube distal end part is thicker than a thickness of the other parts.

According to a form of the present invention, it is preferable that the tube distal end part is coated with a heat-shrinkable tube.

According to a form of the present invention, it is preferable that the tube distal end part is coated with a reinforcing tube.

According to a form of the present invention, it is preferable that the forceps tube has, in order from the distal end side, a first tube, and a second tube connected to a proximal end side of the first tube, the metal element wire is wound only around the second tube between the first tube and the second tube, the first tube is disposed at least at a position facing the opening portion of the shield member, and the first tube and the second tube are connected inside the bending part or inside the distal end hard part.

According to a form of the present invention, it is preferable that bending rigidity of the first tube is higher than bending rigidity of the second tube.

According to a form of the present invention, it is preferable that the metal element wire is wound around the tube distal end part and the tube distal end part is coated with a first insulating tube.

According to a form of the present invention, it is preferable that the metal element wire is incorporated inside an outer shell of the forceps tube, and the tube distal end part is coated with a second insulating tube.

According to a form of the present invention, it is preferable that at least a proximal end-side portion including the region facing the opening portion of the shield member in the forceps pipe is coated with a third insulating tube.

According to the present invention, it is possible to achieve both improvement of the kink resistance of the forceps tube in the bending part and improvement of electromagnetic wave shieldability in the distal end hard part.

### BRIEF DESCRIPTION OF THE DRAWINGS

FIG. 1 is a schematic configuration diagram showing an example of the configuration of an ultrasonography system using an ultrasound endoscope.

FIG. 2 is a partial enlarged perspective view showing the appearance of an example of a distal end part of the ultrasound endoscope shown in FIG. 1.

FIG. 3 is a longitudinal sectional view of the distal end part of the ultrasound endoscope shown in FIG. 2.

FIG. 4 is a sectional view schematically showing the configuration of an example of a coaxial cable.

FIG. 5 is a sectional view schematically showing an example of a signal wire bundle configured with a plurality of coaxial cables.

FIG. 6 is a sectional view of a forceps tube showing a first aspect of increasing bending rigidity of a forceps tube.

FIG. 7 is a sectional view of a forceps tube showing a second aspect of increasing bending rigidity of a forceps tube.

FIG. 8 is a sectional view of a forceps tube showing a third aspect of increasing bending rigidity of a forceps tube.

FIG. 9 is a sectional view of a forceps tube showing a fourth aspect of increasing bending rigidity of a forceps tube.

FIG. 10 is a sectional view of a forceps tube showing a fifth aspect of increasing bending rigidity of a forceps tube.

FIG. 11 is a sectional view of a forceps tube showing a second form of a forceps tube.

FIG. 12 is a sectional view of a forceps tube showing a third form of a forceps tube.

FIG. 13 is a sectional view of a forceps tube showing a fourth form of a forceps tube.

FIG. 14 is a sectional view of a forceps tube showing a fifth form of a forceps tube.

### DESCRIPTION OF THE PREFERRED EMBODIMENTS

Hereinafter, a preferred embodiment of an ultrasound endoscope according to the present invention will be described referring to the accompanying drawings.

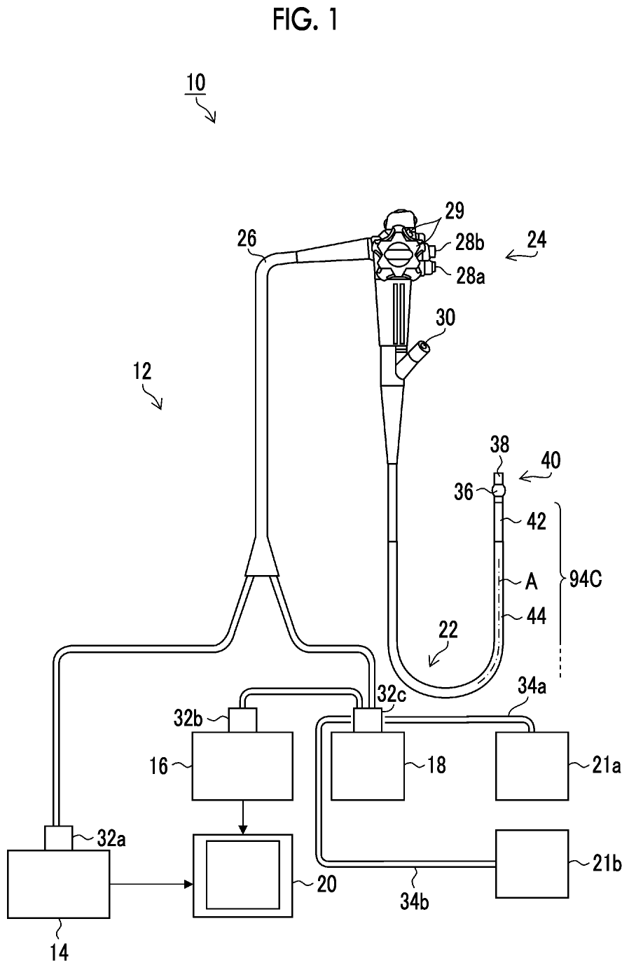

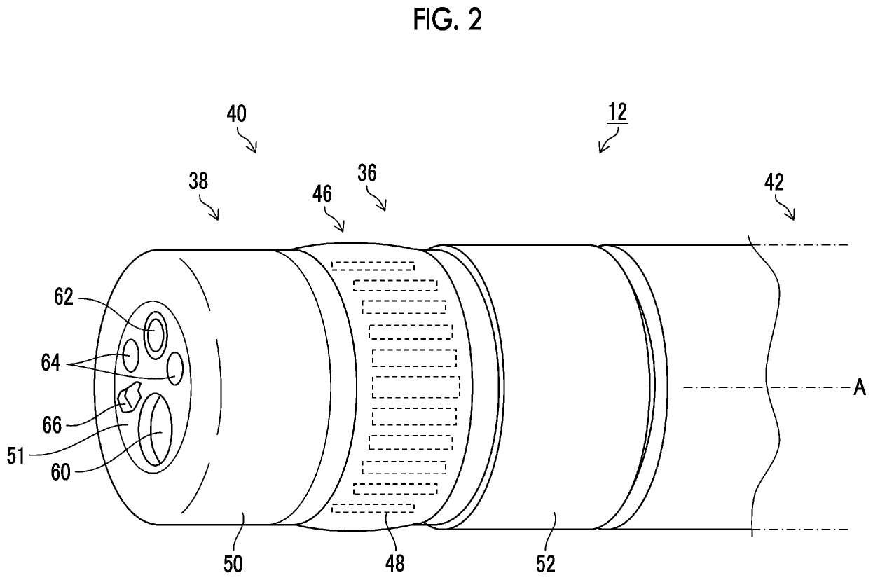

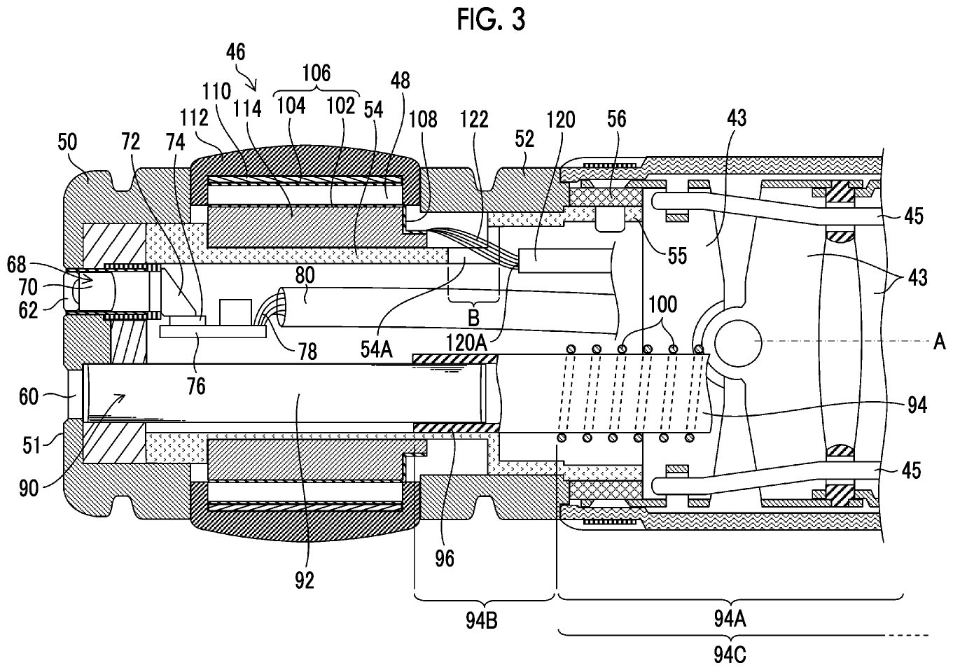

FIG. 1 is a schematic configuration diagram showing an example of an ultrasonography system 10 that uses an ultrasound endoscope 12 of an embodiment. FIG. 2 is a partial enlarged perspective view showing the appearance of an example of a distal end part of the ultrasound endoscope shown in FIG. 1. FIG. 3 is a longitudinal sectional view along a central axis of the distal end part of the ultrasound endoscope shown in FIG. 2.

As shown in FIG. 1, the ultrasonography system 10 comprises the ultrasound endoscope 12, an ultrasound processor device 14 that generates an ultrasound image, an endoscope processor device 16 that generates an endoscope image, a light source device 18 that supplies illumination light, with which the inside of a body cavity is illuminated, to the ultrasound endoscope 12, and a monitor 20 that displays the ultrasound image and the endoscope image. The ultrasonography system 10 comprises a water supply tank 21a that stores cleaning water or the like, and a suction pump 21b that sucks aspirates inside the body cavity.

The ultrasound endoscope 12 has an insertion part 22 that is inserted into the body cavity of the subject, an operating part 24 that is consecutively provided in a proximal end portion of the insertion part 22 and is used by an operator to perform an operation, and a universal cord 26 that has one end connected to the operating part 24.

In the operating part 24, an air/water supply button 28a that opens and closes an air/water supply pipe line (not shown) from the water supply tank 21a, and a suction button 28b that opens and closes a suction pipe line (not shown) from the suction pump 21b are provided side by side. In the operating part 24, a pair of angle knobs 29 and 29 and a treatment tool insertion port 30 are provided.

In the other end portion of the universal cord 26, an ultrasound connector 32a that is connected to the ultrasound processor device 14, an endoscope connector 32b that is connected to the endoscope processor device 16, and a light source connector 32c that is connected to the light source device 18 are provided. The ultrasound endoscope 12 is attachably and detachably connected to the ultrasound processor device 14, the endoscope processor device 16, and the light source device 18 respectively through the connectors 32a, 32b, and 32c. The connector 32c comprises an air/water supply tube 34a that is connected to the water supply tank 21a, and a suction tube 34b that is connected to the suction pump 21b.

The insertion part 22 has, in order from a distal end side, a distal end hard part 40 that has an ultrasound observation part 36 and an endoscope observation part 38, a bending part 42 that is connected to a proximal end side of the distal end hard part 40, and a soft part 44 that connects a proximal end side of the bending part 42 and a distal end side of the operating part 24. The distal end hard part 40, the bending part 42, and the soft part 44 are disposed along a longitudinal axis A of the insertion part 22. The bending part 42 is made by connecting a plurality of bending pieces (angle rings) and is configured to be freely bent. The soft part 44 is slender and long, and has flexibility.

The bending part 42 is remotely bent and operated by rotationally moving and operating a pair of angle knobs 29 and 29 provided in the operating part 24. With this, the distal end hard part 40 can be directed in a desired direction. FIG. 3 shows a plurality of bending pieces 43 configuring the bending part 42, and a plurality of (in FIG. 3, two) bending operating wires 45 of which a distal end side is connected to the bending part 42 and a proximal end side is connected to a pair of angle knobs 29 and 29 (see FIG. 1).

The ultrasound processor device 14 shown in FIG. 1 generates and supplies an ultrasound signal for making a plurality of ultrasound oscillators 48 of the ultrasound transducer 46 (see FIG. 2) configuring the ultrasound observation part 36 generate ultrasonic waves. The ultrasound processor device 14 receives and acquires an echo signal reflected from an observation target part irradiated with the ultrasonic wave, by the ultrasound oscillator 48 and executes various kinds of signal processing on the acquired echo signal to generate an ultrasound image. The generated ultrasound image is displayed on the monitor 20.

The endoscope processor device 16 receives and acquires an image signal acquired from the observation target part illuminated with illumination light from the light source device 18 in the endoscope observation part 38 and executes various kinds of signal processing and image processing on the acquired image signal to generate an endoscope image. The generated endoscope image is displayed on the monitor 20.

The ultrasound processor device 14 and the endoscope processor device 16 are configured of two devices (computers) provided separately. Note that the present invention is not limited thereto, and both the ultrasound processor device 14 and the endoscope processor device 16 may be configured of one device.

The light source device 18 generates illumination light, such as white light consisting of light of three primary colors of red light, green light, and blue light or light of a specific wavelength. The illumination light propagates through a light guide (not shown) in the ultrasound endoscope 12 and is emitted from the endoscope observation part 38 to illuminate an observation target part inside the body cavity.

The monitor 20 receives video signals generated by the ultrasound processor device 14 and the endoscope processor device 16 and displays an ultrasound image and an endoscope image. In regard to the display of the ultrasound image and the endoscope image, only one image may be appropriately switched and displayed on the monitor 20 or both images may be displayed simultaneously.

In the example, although the ultrasound image and the endoscope image are displayed on one monitor 20, a monitor for ultrasound image display and a monitor for endoscope image display may be provided separately. Alternatively, the ultrasound image and the endoscope image may be displayed in a display form other than the monitor 20, for example, in a form of being displayed on a display of a terminal carried with the operator.

Next, the configuration of the distal end hard part 40 will be described referring to FIGS. 2 and 3. As shown in FIG. 2, the distal end hard part 40 is provided with the endoscope observation part 38 that acquires the endoscope image, on the distal end side, and the ultrasound observation part 36 that acquires the ultrasound image, on the proximal end side.

The distal end hard part 40 comprises a cap-shaped distal end component 50 that covers a portion of the endoscope observation part 38 on the distal end side, and a proximal end-side ring 52 that is disposed on the proximal end side of the ultrasound observation part 36 on the proximal end side. The distal end component 50 and the proximal end-side ring 52 consist of an insulating member, such as rigid resin, and serve as an exterior member.

As shown in FIG. 3, a shield ring 54 is connected to the proximal end side of the distal end component 50. A connecting piece 55 is formed on a proximal end side of the shield ring 54, and the connecting piece 55 is connected to the bending piece 43 disposed on the distal end side through an insulating heat-conducting member 56. A forceps channel 90 described below is disposed on one side (inside) with respect to an outer peripheral wall of the shield ring 54, and the ultrasound transducer 46 is disposed on the other side (outside) with respect to the outer peripheral wall of the shield ring 54. In other words, the shield ring 54 is disposed between the ultrasound transducer 46 and the forceps channel 90. The shield ring 54 functions as a shield member of the present invention and suppresses electromagnetic waves emitted from the ultrasound transducer 46. The shield ring 54 will be described below.

Returning to FIG. 2, the endoscope observation part 38 includes a treatment tool outlet port 60 that is opened on a distal end surface 51 of the distal end component 50, observation window 62, illumination windows 64, a cleaning nozzle 66, and the like. Two illumination windows 64 are provided while sandwiching the observation window 62.

The ultrasound observation part 36 is configured with the ultrasound transducer 46. The ultrasound transducer 46 is configured by arranging a plurality of ultrasound oscillators 48 in a peripheral direction of the outer peripheral wall of the shield ring 54.

Inside the distal end hard part 40, a balloon (not shown) into which an ultrasonic wave transmission medium (for example, water or oil) covering the ultrasound observation part 36 is injected may be attachably and detachably fitted. The ultrasonic waves and the echo signals are attenuated in the air. For this reason, the balloon is expanded by injecting the ultrasonic wave transmission medium into the balloon, and is brought into contact with the observation target part, whereby it is possible to eliminate air from a region between the ultrasound transducer 46 of the ultrasound observation part 36 and the observation target part, and to restrain attenuation in the ultrasonic waves and the echo signals.

As shown in FIG. 3, in the distal end hard part 40, an observation system unit 68 is disposed rearward of the observation window 62 (proximal end side). The observation system unit 68 includes an objective lens 70, a prism 72, an imaging element 74, a substrate 76, signal cables 78, and the like.

Reflected light of the observation target part incident from the observation window 62 is taken in by the objective lens 70. An optical path of the taken-in reflected light is folded at a right angle by the prism 72, and the reflected light forms an image on an imaging surface of the imaging element 74. The imaging element 74 photoelectrically converts the reflected light of the observation target part that has formed the image on the above-described imaging surface to output an image signal. Examples of the imaging element 74 include a charge coupled device (CCD) and a complementary metal oxide semiconductor (CMOS).

The imaging element 74 is mounted on the substrate 76. A circuit pattern (not shown) that is electrically connected to the imaging element 74 is formed on the substrate 76. The circuit pattern comprises a plurality of electrodes in an end portion, and a plurality of signal cables 78 are connected to a plurality of electrodes, respectively. The signal cables 78 may be configured of cables in which a core wire is coated with an insulating tube. A plurality of signal cables 78 are inserted into the operating part 24 from the bending part 42 through the soft part 44 shown in FIG. 1 in a state of a shielded cable (signal wire bundle) 80 including a plurality of signal cables 78. Then, a plurality of signal cables 78 are inserted into the universal cord 26 from the operating part 24 and are connected to the endoscope connector 32b. The endoscope connector 32b is connected to the endoscope processor device 16.

Returning to FIG. 3, the forceps channel 90 described above is connected to the treatment tool outlet port 60. The forceps channel 90 is inserted into the insertion part 22 (see FIG. 1), and a distal end side is opened on the distal end surface 51 of the distal end hard part 40.

The forceps channel 90 has a metallic forceps pipe 92 that is disposed inside the shield ring 54, and a forceps tube 94 that is connected to a proximal end side of the forceps pipe 92 inside the shield ring 54. A connection portion (a portion of the forceps pipe 92 coated with the forceps tube 94) 96 of the forceps pipe 92 and the forceps tube 94 is disposed on the proximal end side of the distal end hard part 40 inside the shield ring 54. Here, the forceps channel 90 corresponding to a forceps channel of the present invention. The forceps pipe 92 corresponds to a forceps pipe of the present invention and is formed of steel use stainless (SUS). The forceps tube 94 corresponds to a first form of a forceps tube of the present invention and is formed of a material that is hardly influenced by electromagnetic waves, for example, resin, such as fluororubber or silicon rubber. Here, the material that is hardly influenced by the electromagnetic waves indicates a material that does not obstruct the use of the ultrasound endoscope 12 even though there is the influence of the electromagnetic waves, and includes a material that is completely not influenced by electromagnetic waves.

The forceps tube 94 extends from the inside of the shield ring 54 to a proximal end side of the soft part 44 (see FIG. 1) through the inside of the bending part 42, and a proximal end of the forceps tube 94 is connected to the treatment tool insertion port 30 (see FIG. 1) of the operating part 24. A treatment tool, such as forceps, is inserted into the forceps tube 94 from the treatment tool insertion port 30 and is led out from the treatment tool outlet port 60 through the forceps pipe 92. With this, treatment of the subject is performed by the treatment tool.

The forceps tube 94 comprises a metal element wire 100 that reinforces the forceps tube 94 to suppress kink of the forceps tube 94. The metal element wire 100 corresponds to a metal element wire of the present invention. The metal element wire 100 will be described below.

An emission end of the light guide (not shown) is connected to the illumination windows 64 of the FIG. 2. The light guide extends from the insertion part 22 to the operating part 24 shown in FIG. 1 and is inserted into the universal cord 26 from the operating part 24, and an incidence end of the light guide is connected to the light source connector 32c. The light source connector 32c is connected to the light source device 18. Illumination light emitted from the light source device 18 propagates through the light guide, and a part to be observed is irradiated with the illumination light from the illumination windows 64 of FIG. 2.

An air/water supply channel (not shown) is connected to the cleaning nozzle 66. The air/water supply channel extends from the insertion part 22 to the operating part 24 shown in FIG. 1 and is inserted into the universal cord 26 from the operating part 24. The air/water supply channel is connected to the light source connector 32c and is connected to the water supply tank 21a through the air/water supply tube 34a. To clean the surfaces of the observation window 62 and the illumination windows 64, the cleaning nozzle 66 ejects air or cleaning water from the water supply tank 21a toward the observation window 62 and the illumination windows 64 through the air/water supply channel in the ultrasound endoscope 12.

Hereinafter, the ultrasound transducer 46 will be described. As shown in FIG. 2, the ultrasound transducer 46 is an array of a plurality of channels (CH) consisting of a plurality of ultrasound oscillators 48, for example, 48 to 192 rectangular parallelepiped ultrasound oscillators 48 arranged in a cylindrical shape. In the ultrasound transducer 46, as an example, a plurality of ultrasound oscillators 48 are arranged at predetermined pitches in a peripheral direction like the example shown in the drawing. In this way, the ultrasound oscillators 48 configuring the ultrasound transducer 46 are arranged at regular intervals in a cylindrical shape around a central axis (the longitudinal axis A of the insertion part 22) of the distal end hard part 40. The ultrasound oscillators 48 are sequentially driven based on a drive signal input from the ultrasound processor device 14. Thus, radial electronic scanning is performed with a range in which the ultrasound oscillators 48 are arranged, as a scanning range.

As shown in FIG. 3, the ultrasound transducer 46 includes an electrode part 106 that comprises a plurality of individual electrodes 102 corresponding to a plurality of ultrasound oscillators 48 and a common electrode 104 common to a plurality of ultrasound oscillators 48, a flexible print substrate 108 to which each of a plurality of individual electrodes 102 is connected, and a shield ring 54 that supports a plurality of ultrasound oscillators 48 by an outer peripheral wall. The flexible print substrate 108 is also referred to as a flexible printed circuit (FPC).

The flexible print substrate 108 is thin and flexible, and thus, can be easily bent. Instead of the flexible print substrate 108, a rigid substrate that is not flexible and has high rigidity can be applied. In a case where the flexible print substrate 108 and the rigid substrate are included, simply referred to as a substrate.

The ultrasound transducer 46 further has an acoustic matching layer 110 laminated on the ultrasound oscillators 48, and an acoustic lens 112 laminated on the acoustic matching layer 110. The ultrasound transducer 46 consists of a laminate of the acoustic lens 112, the acoustic matching layer 110, the ultrasound oscillators 48, and a backing material layer 114. The laminate is supported on an outer peripheral wall of the shield ring 54 by a method, such as fitting.

The acoustic matching layer 110 is provided for taking acoustic impedance matching between the subject, such as a human body, and the ultrasound oscillators 48.

The acoustic lens 112 is provided for converging the ultrasonic waves emitted from the ultrasound oscillators 48 toward the observation target part. The acoustic lens 112 consists of, for example, silicon-based resin (millable type silicon rubber, liquid silicon rubber, or the like), butadiene-based resin, or polyurethane-based resin. To increase the transmittance of the ultrasonic waves, powder, such as titanium oxide, alumina, or silica, is mixed in the acoustic lens 112 as needed.

The flexible print substrate 108 that is attached to a side surface on the proximal end side of the backing material layer 114 is electrically connected to a plurality of individual electrodes 102 of the electrode part 106, and are connected to a plurality of coaxial cables 122 accommodated in an ultrasonic wave shielded cable 120. Thus, the individual electrode 102 and each coaxial cable 122 are electrically connected, and as a result, each ultrasound oscillator 48 and the ultrasonic wave shielded cable 120 are electrically connected.

A plurality of coaxial cables 122 are inserted into the operating part 24 from the bending part 42 through the soft part 44 shown in FIG. 1 in a state of being accommodated in the ultrasonic wave shielded cable 120. Then, a plurality of coaxial cables 122 are inserted into the universal cord 26 from the operating part 24 and are connected to the ultrasound connector 32a. The ultrasound connector 32a is connected to the ultrasound processor device 14. Here, the ultrasonic wave shielded cable 120 corresponds to an ultrasonic wave shielded cable of the present invention, and a plurality of coaxial cables 122 correspond to a plurality of signal wires of the present invention.

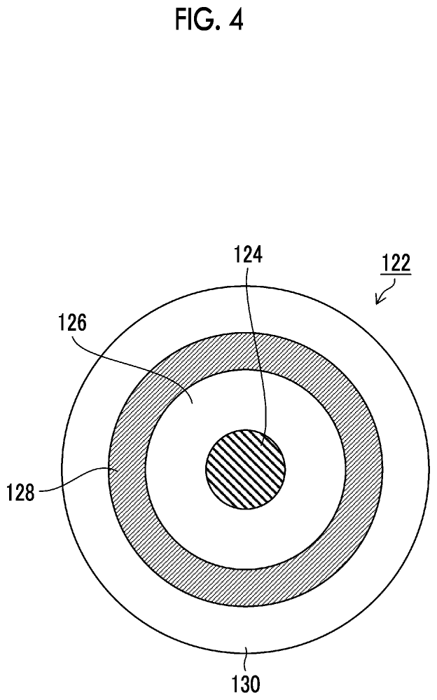

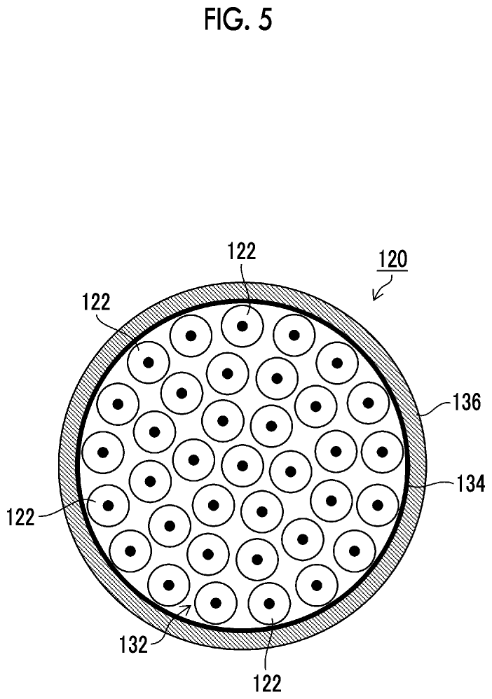

Next, the structures of the coaxial cable 122 and the ultrasonic wave shielded cable 120 will be described based on FIGS. 4 and 5.

As shown in FIG. 4, the coaxial cable 122 comprises a core wire 124 at the center, a first insulating layer 126 on the outer periphery of the core wire 124, a shield member 128 on the outer periphery of the first insulating layer 126, and a second insulating layer 130 on the outer periphery of the shield member 128. The coaxial cable 122 is configured by laminating the core wire 124, the first insulating layer 126, the shield member 128, and the second insulating layer 130 in a concentric circular shape from the center side.

As shown in FIG. 5, the ultrasonic wave shielded cable 120 comprises a cable bundle 132 configured of a plurality of coaxial cables 122, a shield layer 134 with which the cable bundle 132 is coated, and an outer coat 136 with which the shield layer 134 is coated. The cable bundle 132 may be configured by stranding a plurality of coaxial cables 122. The ultrasonic wave shielded cable 120 is handled as one signal wire bundle including a plurality of coaxial cables 122 inside.

The shield layer 134 may be configured by, for example, braiding a plurality of element wires. The element wire is made of a copper wire, a copper alloy wire, or the like subjected to plating processing (tin plating or silver plating).

A tape wound layer (not shown) may be disposed on the outer periphery of the cable bundle 132 inside the shield layer 134. The tape wound layer is, for example, a resin tape and can suppress separation of the cable bundle 132 into the individual coaxial cables 122. In this case, a range of the tape wound layer is basically the same as a range in a longitudinal axis A direction (see FIG. 3) in which the cable bundle 132 is bound.

As shown in FIG. 3, the ultrasonic wave shielded cable 120 configured as above extends from the inside of the bending part 42 to the inside of the distal end hard part 40, and a distal end part 120A of the ultrasonic wave shielded cable 120 is disposed on the inside of the shield ring 54. Then, a plurality of coaxial cables 122 extend from the distal end part 120A of the ultrasonic wave shielded cable 120 to the distal end side. A plurality of extending coaxial cables 122 are wired from the inside (one side to be a side on which the forceps channel 90 is disposed; the same applies hereinafter) of the shield ring 54 to the outside (the other side to be a side on which the ultrasound transducer 46 is disposed; the same applies hereinafter) of the shield ring 54 through the opening portion 54A formed in the shield ring 54 and is connected to the flexible print substrate 108. Here, the opening portion 54A formed in the shield ring 54 corresponds to “an opening portion formed in a shield member” of the present invention.

The shield ring 54 has a function of suppressing electromagnetic waves emitted from the ultrasound transducer 46 as described above. Then, since the shield ring 54 has the above-described function, the entire shield ring 54 is formed of metal, such as SUS, as an example. The shield ring 54 is not limited to the above-described configuration, and for example, a surface of a ring-shaped substrate formed of rigid resin may be coated with a metal film.

Since the opening portion 54A that communicates the inside and the outside of the shield ring 54 is formed in the shield ring 54, it is considered that the above-described suppression function is degraded in the opening portion 54A. In the opening portion 54A, a plurality of coaxial cables 122 that emit electromagnetic waves are wired. For this reason, in the distal end hard part 40, electromagnetic waves may be emitted from the opening portion 54A to the inside of the shield ring 54. In such a configuration, in a case where a metallic member is provided inside the shield ring 54, in particular, in a region B (hereinafter, referred to as “an opening portion facing region B”) facing the opening portion 54A, the metallic member is easily influenced by electromagnetic waves. Thus, there is a need to improve shieldability against electromagnetic waves. Here, the opening portion facing region B corresponds to “a region facing an opening portion” of the present invention. The opening portion facing region B is a region overlapping the opening portion 54A, for example, as viewed from an opening direction (the upper side of FIG. 3) of the opening portion 54A.

On the other hand, since the forceps tube 94 is disposed inside the bending part 42 and is bent with a bending operation of the bending part 42, there is a need to suppress kink that occurs due to the bending operation. For this reason, the forceps tube 94 comprises the metal element wire 100 for improving kink resistance, and the metal element wire 100 is wound in a helical shape around an outer surface of the forceps tube 94 as an example. In a case where the metal element wire 100 that is a metallic member is provided in the opening portion facing region B, since the metal element wire 100 is influenced by electromagnetic waves, the use of the ultrasound endoscope 12 may be obstructed. Accordingly, the ultrasound endoscope 12 of the embodiment employs a configuration described below, thereby achieving both improvement of the kink resistance of the forceps tube 94 and improvement of electromagnetic wave shieldability.

That is, as shown in FIG. 3, the forceps tube 94 is configured in such a manner that the metal element wire 100 is wound around an outer peripheral surface of a tube bending part 94A in the forceps tube 94 (corresponding to “at least a part inside the bending part” of the present invention) disposed inside the bending part 42, and a tube distal end part 94B including the opening portion facing region B is formed of a material that is hardly influenced by electromagnetic waves. Then, as an example of the configuration of the tube distal end part 94B, the forceps tube 94 of the first form has a configuration in which the tube distal end part 94B is configured of only the forceps tube 94. That is, a configuration is made in which the metal element wire 100 that is easily influenced by electromagnetic waves is not wound around the tube distal end part 94B, and resin, such as fluororubber or silicon rubber, is exposed from the opening portion facing region B.

With the forceps tube 94 of the first form, since the metal element wire 100 is wound around the outer peripheral surface of the tube bending part 94A, the kink resistance of the forceps tube 94 in the bending part 42 is improved. Since the tube distal end part 94B including the opening portion facing region B is formed of the material that is hardly influenced by electromagnetic waves, it is possible to reduce the influence of electromagnetic waves from the coaxial cables 122 that are emitted from the opening portion 54A to the inside of the shield ring 54. Accordingly, with the ultrasound endoscope 12 of the embodiment having the forceps tube 94 of the first form, it is possible to achieve both improvement of the kink resistance of the forceps tube 94 in the bending part 42 and improvement of electromagnetic wave shieldability in the distal end hard part 40.

Here, the tube distal end part 94B may have a length corresponding to at least a total length of the opening portion facing region B in the longitudinal axis A direction. That is, the tube distal end part 94B may have a length extending from the opening portion facing region B to the distal end side by a predetermined length or may have a length extending from the opening portion facing region B to the proximal end side by a predetermined length. The tube distal end part 94B illustrated in FIG. 3 has a length extending from the opening portion facing region B to the distal end part of the forceps tube 94 and a length extending from the opening portion facing region B to the proximal end side by a predetermined length. With this, the metal element wire 100 is not present on a distal end side of the opening portion facing region B and the metal element wire 100 can be separated from a proximal end side of the opening portion facing region B, it is possible to further reduce the influence of the electromagnetic waves from the coaxial cables 122. The tube distal end part 94B can be configured by, for example, removing the metal element wire 100 wound around the outer peripheral surface of the tube distal end part 94B in advance, from the tube distal end part 94B. In the tube distal end part 94B, the metal element wire may not be wound around the tube distal end part 94B at the time of manufacturing of the forceps tube 94, and the metal element wire 100 may be wound around a predetermined region (in this example, the tube bending part 94B) of a tube proximal end-side portion excluding the tube distal end part 94B. Here, the tube proximal end-side portion corresponds to “other portions” of the present invention.

Although an example where the forceps tube 94 of the first form has a configuration in which the metal element wire 100 is wound around the tube bending part 94A has been described, the present invention is not limited thereto. That is, the metal element wire 100 may be provided in at least the tube bending part 94A in the tube proximal end-side portion excluding the tube distal end part 94B, and specifically, may be provided in only the tube bending part 94A. With this, the kink resistance of the forceps tube 94 in the bending part 42 is improved. The metal element wire 100 may be provided in a partial region of the tube proximal end-side portion including the tube bending part 94A or may be provided in the entire region of the tube proximal end-side portion. With this, the kink resistance of the forceps tube 94 in the bending part 42 and the soft part 44 is improved.

Incidentally, in a case where the forceps tube 94 is bent in conjunction with the bending operation of the bending part 42, stress (tensile stress and compressive stress) is likely to occur in the tube distal end part 94B connected to the metallic forceps pipe 92. For this reason, the tube distal end part 94B may be damaged due to fatigue. Accordingly, to suppress the above-described damage, it is preferable that the bending rigidity of the tube distal end part 94B is higher than the bending rigidity of the tube proximal end-side portion in the forceps tube 94 excluding the tube distal end part 94B. Hereinafter, a configuration of increasing the bending rigidity of the tube distal end part 94B will be described in connection with some aspects. The same or similar members as or to those of the forceps tube 94 shown in FIG. 3 are represented by the same reference numerals. The bending rigidity described below indicates the bending rigidity of a single tube in a case where the metal element wire 100 is not included.

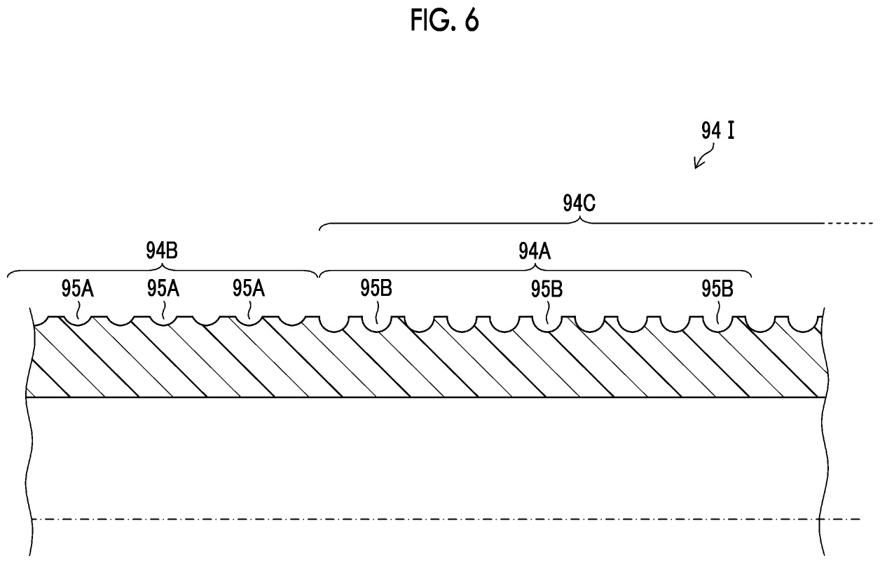

FIG. 6 is a sectional view of a main part of a forceps tube 94I of a first aspect. As shown in FIG. 6, the forceps tube 94I is provided with grooves 95A and 95B for winding metal element wire 100 (see FIG. 3) in both the tube distal end part 94B and a tube proximal end-side portion 94C including the tube bending part 94A, respectively, and the grooves 95A provided in the tube distal end part 94B are shallower than the grooves 95B provided in the tube proximal end-side portion 94C. As such a configuration is employed, with the forceps tube 94I of the first aspect, since the bending rigidity of the tube distal end part 94B is higher than the bending rigidity of the tube proximal end-side portion 94C, it is possible to suppress damage of the tube distal end part 94B due to fatigue.

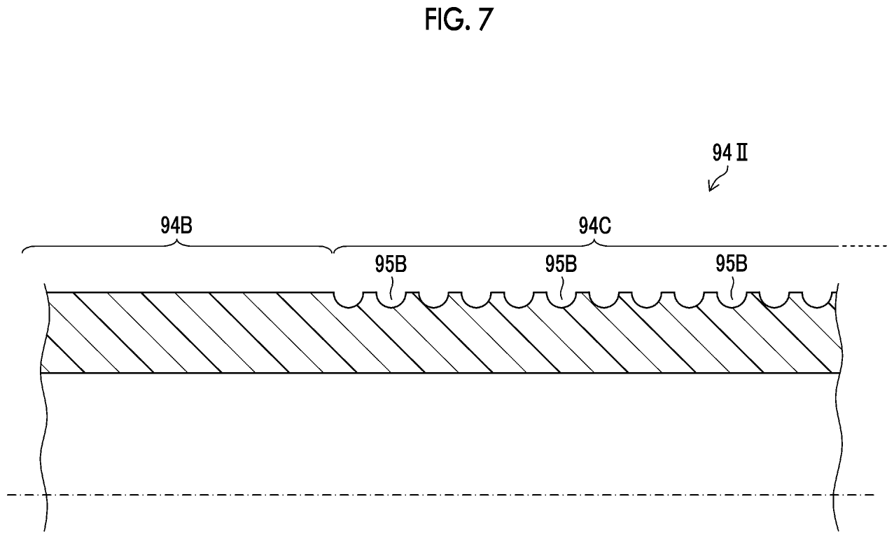

FIG. 7 is a sectional view of a main part of a forceps tube 94II of a second aspect. As shown in FIG. 7, the forceps tube 94II is provided with grooves 95B for winding the metal element wire 100 (see FIG. 3) in only the tube proximal end-side portion 94C. As such a configuration is employed, with the forceps tube 94II of the second aspect, since the bending rigidity of the tube distal end part 94B is higher than the bending rigidity of the tube proximal end-side portion 94C, it is possible to suppress the above-described damage.

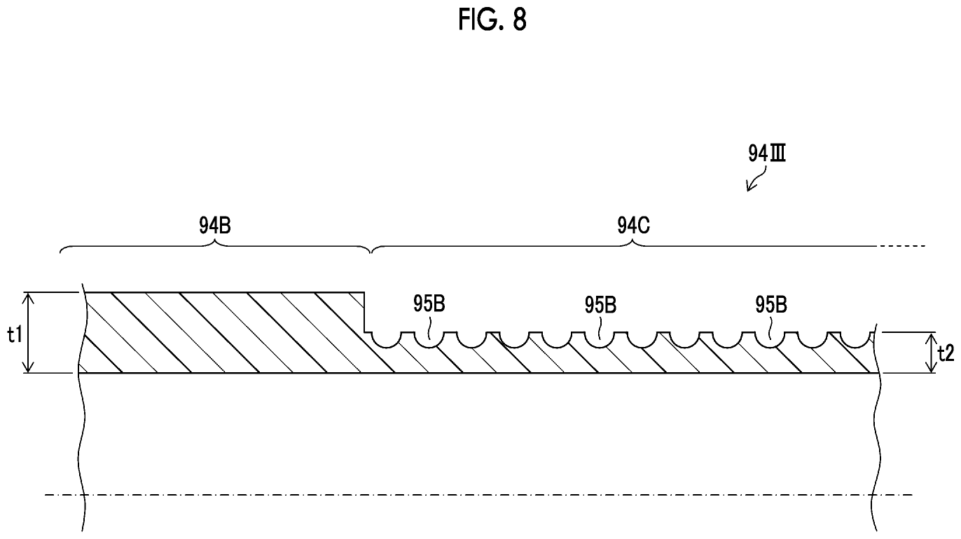

FIG. 8 is a sectional view of a main part of a forceps tube 94III of a third aspect. As shown in FIG. 8, the forceps tube 94III has a configuration in which a thickness t1 of the tube distal end part 94B is thicker than a thickness t2 of the tube proximal end-side portion 94C. In other words, an inner diameter of the tube distal end part 94B is equal to an inner diameter of the tube proximal end-side portion 94C, and an outer diameter of the tube distal end part 94B is greater than an outer diameter of the tube proximal end-side portion 94C. As such a configuration is employed, with the forceps tube 94III of the third aspect, since the bending rigidity of the tube distal end part 94B is higher than the bending rigidity of the tube proximal end-side portion 94C, it is possible to suppress the above-described damage.

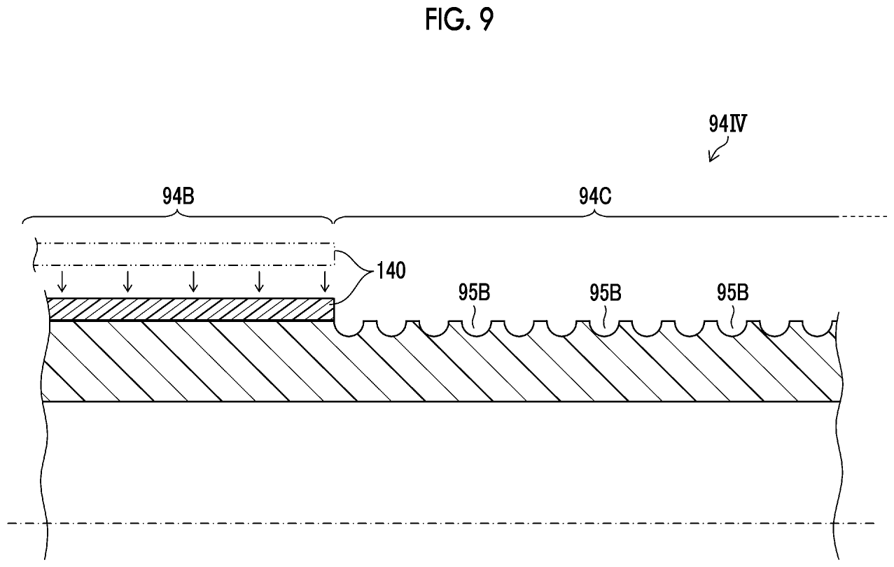

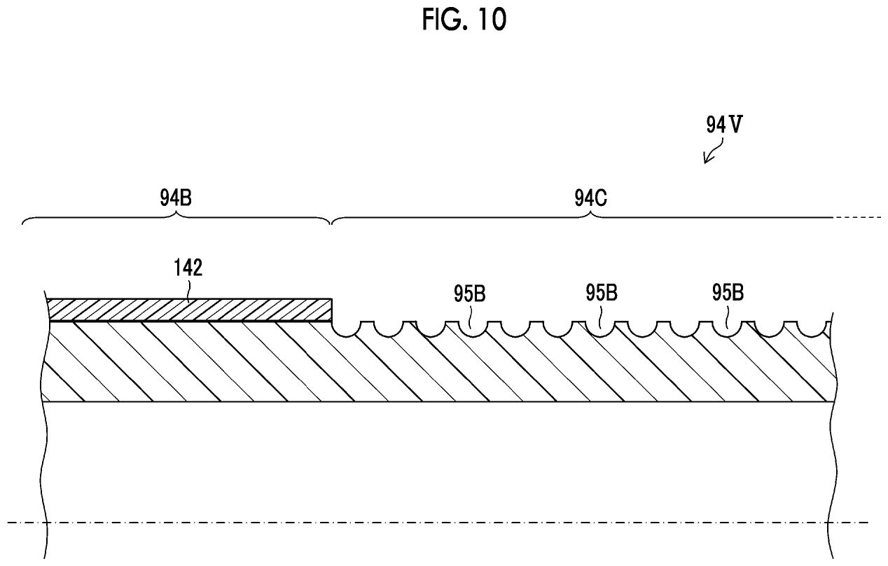

FIG. 9 is a sectional view of a main part of a forceps tube 94IV of a fourth aspect. As shown in FIG. 9, the forceps tube 94IV has a configuration in which a tube distal end part 94B is coated with a heat-shrinkable tube 140 subjected to thermosetting processing. As such a configuration is employed, with the forceps tube 94IV of the fourth aspect, since the bending rigidity of the tube distal end part 94B is higher than the bending rigidity of the tube proximal end-side portion 94C by the thermoset heat-shrinkable tube 140, it is possible to suppress the above-described damage. As the heat-shrinkable tube 140, as an example, heat shrinkable silicon rubber that is hardly influenced by electromagnetic waves can be used. In FIG. 9, although an aspect with no grooves shown in FIG. 7 has been shown as the tube distal end part 94B, the present invention is not limited thereto, and the aspect of the shallow grooves 95A or the aspect of the deep grooves 95B shown in FIG. 6 may be made. In the fourth aspect, although the heat-shrinkable tube 140 has been illustrated as a tube with which the tube distal end part 94B is coated, the present invention is not limited thereto, and the tube distal end part 94B may be coated with tubes of other aspects. An example is shown in FIG. 10.

FIG. 10 is a sectional view of a main part of a forceps tube 94V of a fifth aspect. As shown in FIG. 10, the forceps tube 94V has a configuration in which the tube distal end part 94B is reinforced by coating the tube distal end part 94B with a reinforcing tube 142 of another aspect. As such a configuration is employed, with the forceps tube 94V of the fifth aspect, since the bending rigidity of the tube distal end part 94B is higher than the bending rigidity of the tube proximal end-side portion 94C by the reinforcing tube 142, it is possible to suppress the above-described damage. As the reinforcing tube 142, as an example, silicon rubber that is hardly influenced by electromagnetic waves can be used. In FIG. 10, although an aspect with no grooves shown in FIG. 7 has been shown as the tube distal end part 94B, the present invention is not limited thereto, and the aspect of the shallow grooves 95A or the aspect of the deep grooves 95B shown in FIG. 6 may be applied.

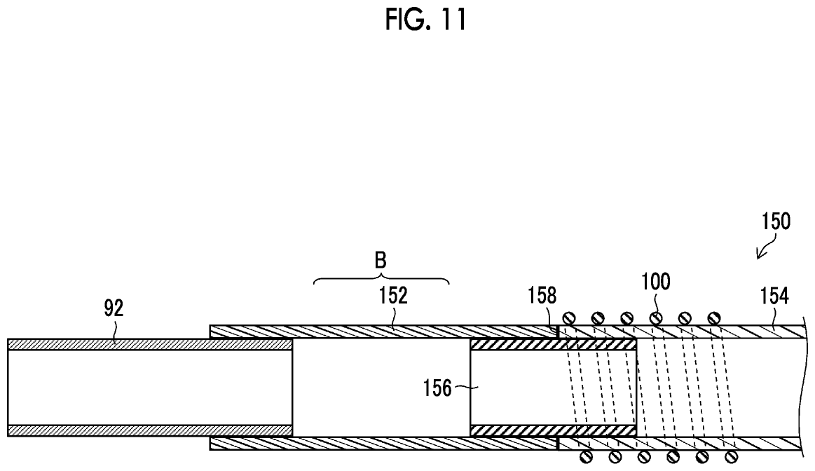

Next, a second form of a forceps tube will be described. FIG. 11 is a sectional view of a forceps tube 150 according to the second form. The same or similar members as or to those of the forceps tube 94 shown in FIG. 3 are represented by the same reference numerals.

A difference between the forceps tube 150 of the second form shown in FIG. 11 and the forceps tube 94 of the first form shown in FIG. 3 will be described. While the forceps tube 94 shown in FIG. 3 has a configuration in which the tube distal end part 94B and the tube proximal end-side portion 94C are configured of one forceps tube 94, in the forceps tube 150 shown in FIG. 11, the forceps tube 150 is configured of a first tube 152, a second tube 154, and the like.

Specifically, the forceps tube 150 has, in order from a distal end side, the first tube 152 and the second tube 154 connected to a proximal end side of the first tube 152. A metal element wire 100 is wound around only the second tube 154 between the first tube 152 and the second tube 154. Then, the first tube 152 is disposed at least at a position (opening portion facing region B) facing the opening portion 54A (see FIG. 3) of the shield ring 54 (see FIG. 3), and the first tube 152 and the second tube 154 are connected inside the bending part 42 (see FIG. 3) as an example. Similarly to the tube distal end part 94B shown in FIG. 3, the first tube 152 is formed of resin that is hardly influenced by electromagnetic waves, such as fluororubber or silicon rubber. The first tube 152 is connected to the proximal end side of the forceps pipe 92.

With the forceps tube 150 of the second form, since the metal element wire 100 is wound around an outer peripheral surface of the second tube 154 disposed inside the bending part 42, the kink resistance of the forceps tube 150 in the bending part 42 (see FIG. 1) is improved. Since the first tube 152 that is hardly influenced by electromagnetic waves is disposed in the opening portion facing region B, it is possible to reduce the influence of the electromagnetic waves from the coaxial cable 122 (see FIG. 3) that are emitted from the opening portion 54A (see FIG. 3) to the inside of the shield ring 54 (see FIG. 3). Accordingly, even in an ultrasound endoscope having the forceps tube 150 of the second form, it is possible to achieve both improvement of the kink resistance of the forceps tube 150 in the bending part 42 and improvement of electromagnetic wave shieldability in the distal end hard part 40.

As an aspect for connecting the first tube 152 and the second tube 154, a pipe 156 for joint can be used as shown in FIG. 11. In this case, the first tube 152 and the second tube 154 can be connected by fitting a distal end side of the pipe 156 to the proximal end side of the first tube 152 and fitting a proximal end side of the pipe 156 to the distal end side of the second tube 154. A coupling portion 158 of the first tube 152 and the second tube 154 is not limited to the inside of the bending part 42 (see FIG. 3), and may be inside the distal end hard part 40 (see FIG. 3). In both cases, the bending operation of the bending part 42 is not obstructed. It is preferable the pipe 156 is formed of flexible rubber as an example. With this, since the pipe 156 is bent to follow a bending operation of the first tube 152 and the second tube 154, the bending part 42 smoothly performs the bending operation. The first tube 152 and the second tube 154 may be directly connected using, for example, an adhesive.

Even in the forceps tube 150 shown in FIG. 11, it is preferable that the bending rigidity of the first tube 152 is higher than the bending rigidity of the second tube 154. As a configuration of increasing the bending rigidity of the first tube 152, the aspect of the shallow grooves 95A shown in FIG. 6, the aspect with no grooves shown in FIG. 7, the aspect of the large thickness shown in FIG. 8, the aspect of using the heat-shrinkable tube 140 shown in FIG. 9, and the aspect of using reinforcing tube 142 shown in FIG. 10 can be employed.

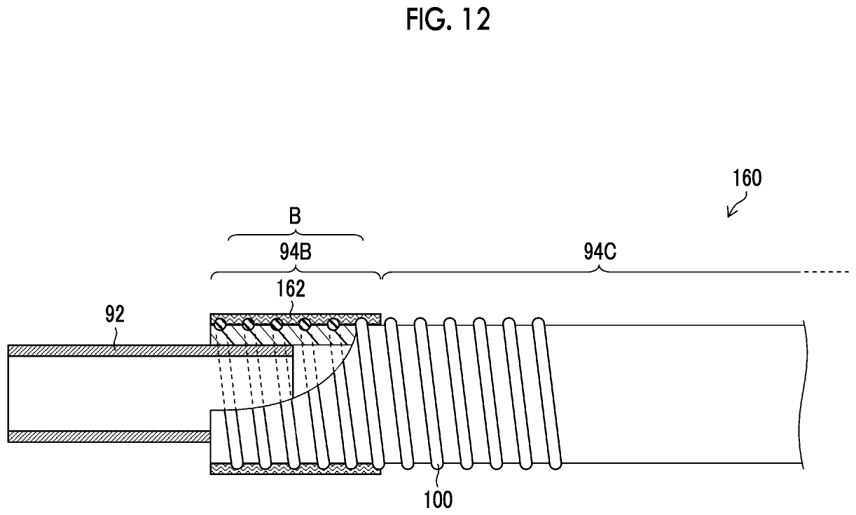

Next, a third form of a forceps tube will be described. FIG. 12 is a sectional view of a forceps tube 160 according to the third form. The same or similar members as or to those of the forceps tube 94 shown in FIG. 3 are represented by the same reference numerals.

A difference between the forceps tube 160 of the third form shown in FIG. 12 and the forceps tube 94 of the first form shown in FIG. 3 will be described. While the forceps tube 94 shown in FIG. 3 has a configuration in which the metal element wire 100 is not wound around the tube distal end part 94B, in the forceps tube 160 shown in FIG. 12, the metal element wire 100 is wound around the tube distal end part 94B, and the tube distal end part 94B is coated with an insulating tube 162. The insulating tube 162 is formed of, for example, resin, such as fluororubber or silicon rubber, and corresponds to a first insulating tube of the present invention.

With the forceps tube 160 of the third form, since the metal element wire 100 is wound around the outer peripheral surface of the tube proximal end-side portion 94C, the kink resistance of the forceps tube 160 in the bending part 42 (see FIG. 1) is improved. Since the tube distal end part 94B coated with the insulating tube 162 is disposed at a position (opening portion facing region B) facing the opening portion 54A (see FIG. 3) of the shield ring 54 (see FIG. 3), it is possible to reduce the electromagnetic waves from the coaxial cable 122 (see FIG. 3) that are emitted from the opening portion 54A to the inside of the shield ring 54. Accordingly, even in an ultrasound endoscope having the forceps tube 160 of the third form, it is possible to achieve both improvement of the kink resistance of the forceps tube 160 in the bending part 42 and improvement electromagnetic wave shieldability in the distal end hard part 40.

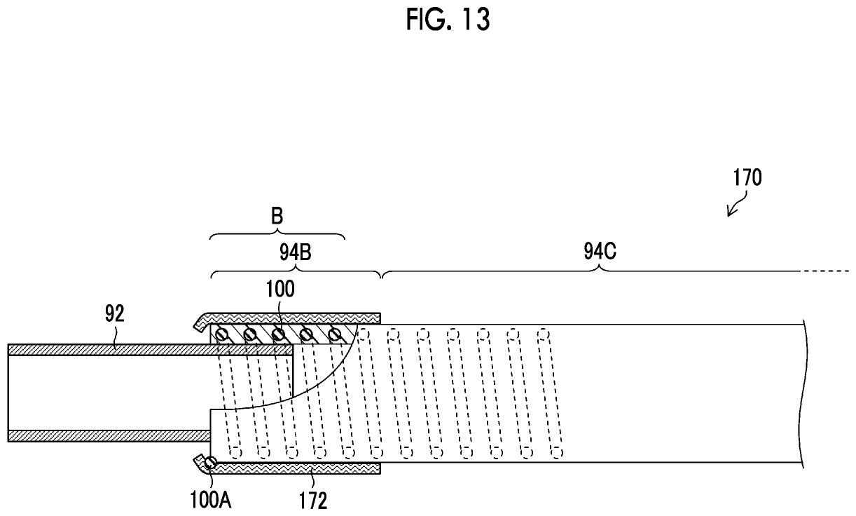

Next, a fourth form of a forceps tube will be described. FIG. 13 is a sectional view of a forceps tube 170 according to the fourth form. The same or similar members as or to those of the forceps tube 94 shown in FIG. 3 are represented by the same reference numerals.

A difference between the forceps tube 170 of the fourth form shown in FIG. 13 and the forceps tube 160 of the third form shown in FIG. 12 will be described. While the forceps tube 160 shown in FIG. 12 has a configuration in which the metal element wire 100 is wound around the outer peripheral surface each of the tube distal end part 94B and the tube proximal end-side portion 94C, in the forceps tube 170 shown in FIG. 13, a metal element wire 100 is incorporated inside an outer shell of each of the tube distal end part 94B and the tube proximal end-side portion 94C, and the tube distal end part 94B is coated with an insulating tube 172. The insulating tube 172 is formed of, for example, resin, such as fluororubber or silicon rubber, and corresponds to a second insulating tube of the present invention.

In the forceps tube 170 of the fourth form, while a form is made in which the metal element wire 100 is incorporated inside the outer shell of the tube distal end part 94B, the metal element wire 100 may be influenced by electromagnetic waves. In such a case, like the forceps tube 170 of the fourth form, the influence of the electromagnetic waves is reduced by coating the tube distal end part 94B with the insulating tube 172.

With the forceps tube 170 of the fourth form, since the metal element wire 100 is incorporated inside the outer shell of the tube proximal end-side portion 94C, the kink resistance of the forceps tube 170 in the bending part 42 (see FIG. 1) is improved. Since the tube distal end part 94B coated with the insulating tube 172 is disposed at a position (opening portion facing region B) facing the opening portion 54A (see FIG. 3) of the shield ring 54 (see FIG. 3), it is possible to reduce the electromagnetic waves from the coaxial cable 122 (see FIG. 3) that are emitted from the opening portion 54A to the inside of the shield ring 54. Accordingly, even in an ultrasound endoscope having the forceps tube 170 of the fourth form, it is possible to achieve both improvement of the kink resistance of the forceps tube 170 in the bending part 42 and improvement electromagnetic wave shieldability in the distal end hard part 40.

In the forceps tube 170 of the fourth form, since a distal end 100A of the metal element wire 100 may be exposed from the distal end of the tube distal end part 94B, as in FIG. 13, the tube distal end part 94B is coated with the insulating tube 172 such that the distal end side is not exposed, whereby it is possible to reduce an influence of electromagnetic waves on the distal end 100A.

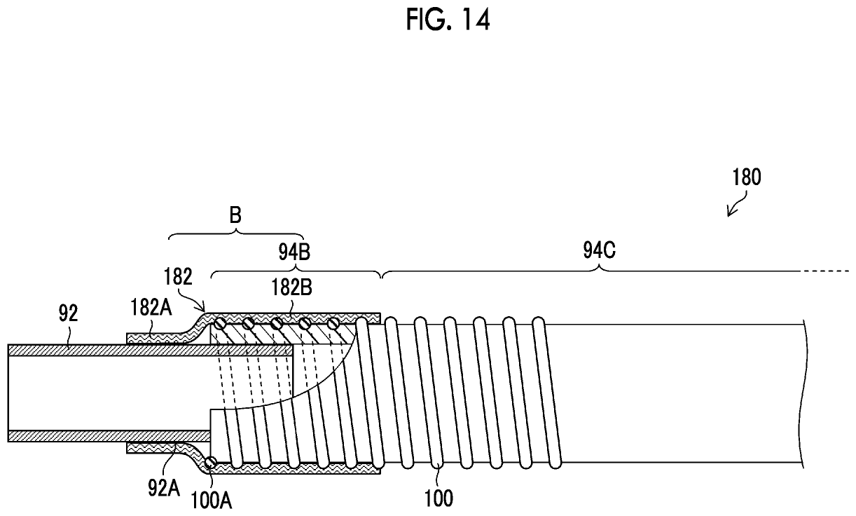

Next, a fifth form of a forceps tube will be described. FIG. 14 is a sectional view of a forceps tube 180 according to the fifth form. The same or similar members as or to those of the forceps tube 160 shown in FIG. 12 are represented by the same reference numerals.

A difference between the forceps tube 180 of the fifth form shown in FIG. 14 and the forceps tube 160 of the third form shown in FIG. 12 will be described. While the forceps tube 160 shown in FIG. 12 has a configuration in which only the tube distal end part 94B is coated with the insulating tube 162, in the forceps tube 180 shown in FIG. 14, at least a proximal end-side portion 92A in a forceps pipe 92 that is positioned in the opening portion facing region B is coated with a distal end-side portion 182A of an insulating tube 182, and the tube distal end part 94B is coated with a proximal end-side portion 182B of the insulating tube 182. The insulating tube 182 is formed of, for example, resin, such as fluororubber or silicon rubber, and corresponds to a third insulating tube of the present invention.

With the forceps tube 180 of the fifth form, since the metal element wire 100 is wound around the outer peripheral surface of the tube proximal end-side portion 94C, the kink resistance of the forceps tube 180 in the bending part 42 (see FIG. 1) is improved. Since the proximal end-side portion 92A in the metallic forceps pipe 92 positioned in the opening portion facing region B and the tube distal end part 94B are coated with the insulating tube 182, it is possible to reduce an influence of electromagnetic waves. Accordingly, even in an ultrasound endoscope having the forceps tube 180 of the fifth form, it is possible to achieve both improvement of the kink resistance of the forceps tube 180 in the bending part 42 and improvement of electromagnetic wave shieldability in the distal end hard part 40.

In FIG. 14, although an example where the insulating tube 182 in which the distal end-side portion 182A and the proximal end-side portion 182B are integrated has been shown, the present invention is not limited thereto, and an insulating tube in which the distal end-side portion 182A and the proximal end-side portion 182B are separated may be employed. In a case where fittability of the insulating tube with respect to the forceps tube is considered, it is preferable that the insulating tube 182 in which the distal end-side portion 182A and the proximal end-side portion 182B are integrated is employed. The distal end-side portion 182A of the insulating tube 182 shown in FIG. 14 can also be applied to the forceps tube 94 of the first form shown in FIG. 3, the forceps tube 150 of the second form shown in FIG. 11, and the forceps tube 170 of the fourth form shown in FIG. 13.

Although the endoscope according to the embodiment has been described above, some improvements or modifications may be made without departing from the spirit and scope of the present invention.

### EXPLANATION OF REFERENCES

10: ultrasonography system
12: ultrasound endoscope
14: ultrasound processor device
16: endoscope processor device
18: light source device
20: monitor
21a: water supply tank
21b: suction pump
22: insertion part
24: operating part
26: universal cord
28a: air/water supply button
28b: suction button
29: angle knob
30: treatment tool insertion port
32a: connector
32b: connector
32c: connector
34a: air/water supply tube
34b: suction tube
36: ultrasound observation part
38: endoscope observation part
40: distal end hard part
42: bending part
43: bending piece
44: soft part
45: bending operating wire
46: ultrasound transducer
48: ultrasound oscillator
50: distal end component
51: distal end surface
52: proximal end-side ring
54: shield ring
54A: opening portion
55: connecting piece
56: insulating heat-conducting member
60: treatment tool outlet port
62: observation window
64: illumination window
66: cleaning nozzle
68: observation system unit
70: objective lens
72: prism
74: imaging element
76: substrate
78: signal cable
80: shielded cable
90: forceps channel
92: forceps pipe
94: forceps tube
94I: forceps tube
94II: forceps tube
94III: forceps tube
94IV: forceps tube
94V: forceps tube
94A: tube bending part
94B: tube distal end part
94C: tube proximal end-side portion
95A: groove
95B: groove
96: connection portion
100: metal element wire
100A: distal end
102: individual electrode
104: common electrode
106: electrode part
108: flexible print substrate
110: acoustic matching layer
112: acoustic lens
114: backing material layer
120: ultrasonic wave shielded cable
120A: distal end part
122: coaxial cable
124: core wire
126: first insulating layer
128: shield member
130: second insulating layer
132: cable bundle
134: shield layer
136: outer coat
140: heat-shrinkable tube
142: reinforcing tube
150: forceps tube
152: first tube
154: second tube
156: pipe
158: coupling portion
160: forceps tube
162: insulating tube
170: forceps tube
172: insulating tube
180: forceps tube
182: insulating tube
182A: distal end-side portion
182B: proximal end-side portion
A: longitudinal axis
B: opening portion facing region

What is claimed is:

1. An ultrasound endoscope comprising:
an insertion part in which a distal end hard part, a bending part connected to a proximal end side of the distal end hard part, and a soft part connected to a proximal end side of the bending part are provided along a longitudinal axis direction;
an ultrasound transducer in which a plurality of ultrasound oscillators configured to transmit and receive ultrasonic waves are arranged along a peripheral direction of the distal end hard part;
a forceps channel that is inserted into the insertion part and has a distal end side opened on a distal end surface of the distal end hard part;
a shield member that is disposed between the ultrasound transducer and the forceps channel, and suppresses electromagnetic waves emitted from the ultrasound transducer;
an ultrasonic wave shielded cable that passes through the bending part from the soft part and has a distal end part disposed on one side to be a side of the shield member on which the forceps channel is disposed; and
a plurality of signal wires that are accommodated in the ultrasonic wave shielded cable, extend from the distal end part of the ultrasonic wave shielded cable, and are connected to the plurality of ultrasound oscillators, respectively,
wherein the shield member has an opening portion for wiring the plurality of signal wires from the one side of the shield member to the other side to be a side on which the ultrasound transducer is disposed,
the forceps channel has a metallic forceps pipe disposed on the one side of the shield member, and a forceps tube that is connected to a proximal end side of the forceps pipe on the one side of the shield member, and
the forceps tube is configured such that a metal element wire is wound around at least a part inside the bending part, and a tube distal end part including a region facing the opening portion of the shield member is formed of a material that is hardly influenced by the electromagnetic waves.

2. The ultrasound endoscope according to claim 1,
wherein the metal element wire is provided in only other parts in the forceps tube except for the tube distal end part.

3. The ultrasound endoscope according to claim 2,
wherein bending rigidity of the tube distal end part is higher than bending rigidity of the other parts.

4. The ultrasound endoscope according to claim 3,
wherein the forceps tube is provided with grooves for winding the metal element wire around the tube distal end part and the other parts, and
the groove provided in the tube distal end part is shallower than a depth of the groove provided in the other parts.

5. The ultrasound endoscope according to claim 3,
wherein, in the forceps tube, a groove for winding the metal element wire is provided in only the other parts.

6. The ultrasound endoscope according to claim 3,
wherein a thickness of the tube distal end part is thicker than a thickness of the other parts.

7. The ultrasound endoscope according to claim 3,
wherein the tube distal end part is coated with a heat-shrinkable tube.

8. The ultrasound endoscope according to claim 3,
wherein the tube distal end part is coated with a reinforcing tube.

9. The ultrasound endoscope according to claim 1,
wherein the forceps tube has, in order from the distal end side, a first tube, and a second tube connected to a proximal end side of the first tube,
the metal element wire is wound only around the second tube between the first tube and the second tube,
the first tube is disposed at least at a position facing the opening portion of the shield member, and
the first tube and the second tube are connected inside the bending part or inside the distal end hard part.

10. The ultrasound endoscope according to claim 9,
wherein bending rigidity of the first tube is higher than bending rigidity of the second tube.

11. The ultrasound endoscope according to claim 1,
wherein the metal element wire is wound around the tube distal end part and the tube distal end part is coated with a first insulating tube.

12. The ultrasound endoscope according to claim 1,
wherein the metal element wire is incorporated inside an outer shell of the forceps tube, and
the tube distal end part is coated with a second insulating tube.

13. The ultrasound endoscope according to claim 1,
wherein at least a proximal end-side portion including the region facing the opening portion of the shield member in the forceps pipe is coated with a third insulating tube.
